(19) United States
(12) Patent Application Publication
(10) Pub. No.: US 2007/0049928 A1
(43) Pub. Date: Mar. 1, 2007

(54) Nickel titanium alloy electrosurgery instrument

(76) Inventors: Richard P. Fleenor, Greenwood Village, CO (US); Robert L. Bromley, Littleton, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 11/348,097

(22) Filed: Feb. 6, 2006

Related U.S. Application Data
(60) Provisional application No. 60/651,172, filed on Feb. 8, 2005.

(51) Int. Cl. A61B 18/14

(52) U.S. Cl. 606/45; 606/49; 606/51

(57) ABSTRACT

An electrosurgical instrument including a Nickel Titanium alloy electrode that reduces eschar build-up during surgical procedures. Electrosurgical instruments conforming to aspects of the invention include electrosurgery blades, laparoscopic electrodes, and electrosurgery forceps including one or more Nickel Titanium alloy electrodes.

### CROSS-REFERENCE TO RELATED APPLICATION

This application is a non-provisional patent application claiming priority of provisional patent application No. 60/651,172 filed Feb. 8, 2005 in the United States Patent and Trademark Office and titled “Nickel Titanium Alloy Electrosurgery Instrument,” which is hereby incorporated by reference herein.

### FIELD OF THE INVENTION

The present invention generally involves the field of electrosurgery instruments, and more particularly involves electrosurgery instruments including a biocompatible Nickel Titanium alloy electrode or other member intended to cut and/or coagulate tissue when appropriately energized.

### BACKGROUND

Electrosurgery involves the application of radio frequency (“RF”) current to tissue. Electrosurgery may be used to cut tissue, to coagulate blood (hemostasis), to both cut and coagulate simultaneously, and to desiccate tissue. One particular type of electrosurgical instrument is an electrosurgery blade, such as the stainless steel electrosurgery blade 10 shown in FIG. 1. For an electrosurgical procedure, the electrosurgical blade is connected to an RF generator. The blade is positioned in a handle, often referred to as a “pencil”, that includes a connection to the generator and switches for energizing the blade. During surgery, a surgeon manipulates the instrument to place an electrode portion 12 of the blade adjacent target tissue and selects characteristics of the RF current to cut and coagulate tissue, and to desiccate tissue. The ability to simultaneously cut and coagulate is one of the many advantages of electrosurgery.

Another type of electrosurgical instrument are the bipolar forceps. Bipolar forceps have two opposing pincers with electrode tips. The distal ends of the pincers are connected, so that a surgeon can squeeze the pincers to grasp tissue between the tips. In a typical procedure, a surgeon manipulates the tips around a target tissue and energizes the tips to dry out the tissue, such as to desiccate a bleeding vessel.

A third type of electrosurgery instrument is a laparoscopic electrode used in laparoscopic surgery. Generally, laparoscopic surgery is a minimally invasive procedure that involves the insertion of a small fiber optic imaging device, referred to as a laparoscope, into a patient through a small incision that allows a surgeon to see a target area in the patient. Through a second small incision a laparoscopic electrode is inserted into the patient, either directly or through a tube often referred to as a cannula. A laparoscopic electrode includes a relatively long insulated shaft with an electrode at its distal end. The proximal end of the shaft includes a plug for connecting the laparoscopic electrode to a generator. Laparoscopic electrodes can take on many shapes.

One drawback to conventional electrosurgery instruments involves the buildup of eschar 14 on the electrode 12 during a procedure. During electrosurgery, cell protein, tissue, fluids and blood contact the surface of the electrode. In many procedures, the electrosurgery current heats the electrode significantly. The temperature of the electrode causes the body material in contact with the electrode to char and form a material often referred to as “eschar.” As eschar builds up, the electrode's performance decreases, eventually becoming unusable. The eschar negatively affects the energy transfer to the target tissue, the maneuverability of the instrument, the ability of the surgeon to view the target tissue, and also causes smoke, as well as other problems. The numerous problems associated with eschar buildup on electrosurgical electrodes are well recognized, and numerous concepts have been introduced to clean eschar from electrodes and reduce eschar buildup on electrodes.

One approach to removing the eschar is for the surgeon to periodically scrape the eschar from the electrode with a tool. Scraping is generally not preferred by surgeons because it does not remove all of the eschar and disrupts the surgical procedure. An approach to reducing eschar buildup on electrodes involves coating the electrode with a material that helps reduce eschar buildup or makes cleaning more effective. Various coating materials have been applied to electrodes, with varying degrees of success. The coating types include fluorinated hydrocarbon materials (e.g., “Teflon”), silicone, ceramics, paralyene polymers, among others. Coatings add cost to the electrode and are not always effective. Further, coating performance can degrade during use due to the high heat and electrical currents in the electrode.

What is needed, amongst other things, is an electrode, and associated instruments, that reduces or eliminates eschar buildup and/or addresses some or all of the deficiencies in existing instruments.

### SUMMARY

One aspect of the invention involves an electrosurgery instrument of various types for conducting electrical energy to tissue during an electrosurgery procedure. The electrosurgery instrument includes a conductive shaft region arranged to receive electrical energy. An electrode is in electrical communication with the conductive shaft. The electrode is fabricated with a Nickel Titanium alloy, which is resistant to eschar buildup during procedures, amongst other advantages.

Another aspect of the invention involves an electrosurgery s including a first pincer including a first Nickel Titanium alloy tip region. The forceps further includes a second pincer including a second Nickel Titanium alloy tip region. The first and second pincers are operably coupled together.

### BRIEF DESCRIPTION OF THE DRAWINGS

FIG. 1 is an isometric view of an electrosurgery blade including a stainless steel electrode with eschar buildup thereon;

FIG. 2 is an isometric view of a prior art electrosurgery blade type electrosurgical instrument with a Nickel Titanium shape memory alloy electrode and an electrosurgery pencil adapted to receive and energize the blade, conforming to aspects of the present invention;

FIG. 3 is a side view of the electrosurgery blade of FIG. 2, conforming to aspects of the present invention;

FIG. 4 is a top view of the electrosurgery blade of FIG. 2, conforming to aspects of the present invention;

FIG. 5 is an isometric view of a second alternative electrosurgery blade with a Nickel Titanium shape memory alloy electrode, conforming to aspects of the present invention;

FIG. 6 is an isometric view of a third alternative electrosurgery blade with a Nickel Titanium shape memory alloy electrode, conforming to aspects of the present invention;

FIG. 7 is an isometric view of a fourth alternative electrosurgery blade with a Nickel Titanium shape memory alloy electrode, conforming to aspects of the present invention;

FIG. 8 is an isometric view of an electrosurgery forceps type electrosurgery instrument with Nickel Titanium shape-memory alloy pincer tips, conforming to aspects of the present invention;

FIG. 9 is an isometric view of a laparoscopic type electrosurgery instrument with a Nickel Titanium alloy electrode, conforming to aspects of the present invention; and

FIG. 10 is an isometric view of a second alternative laparoscopic type electrosurgery instrument with a Nickel Titanium alloy electrode, conforming to aspects of the present invention.

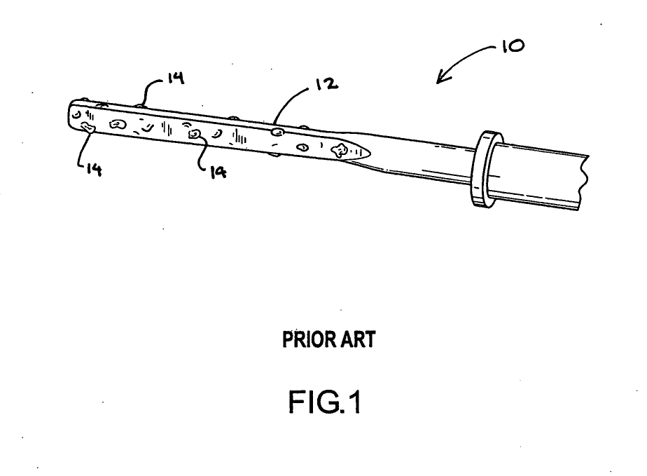

### DETAILED DESCRIPTION OF EMBODIMENTS OF THE INVENTION

Aspects of the present invention involve an electrosurgery instrument employing a Nickel Titanium shape-memory alloy (“NiTi-SMA”) electrode. One particular type of NiTi-SMA employable in embodiments of the invention is Nitinol®. Generally speaking, NiTi-SMA such as Nitinol®, may be originally formed in a first shape, then formed into a second shape, and upon application of heat will return to the original or first shape. The shape memory aspects of NiTi-SMA, however, are not particularly relevant to the present invention. The inventors of the present patent application have discovered that NiTi-SMAs, besides having shape memory characteristics, are also highly resistant to eschar build-up on electrosurgery blades. As such, aspects of the present invention involve an electrosurgery instrument or unit employing a NiTi-SMA electrode for cutting, coagulating, and desiccating tissue using RF current. As used herein, the term “electrode” is meant to refer to the electrosurgical cutting/coagulating area of an electrosurgical instrument, such as the electrode area of an electrosurgery blade, tips of electrosurgery forceps, and the hook, ball, needle, spatula and other shaped laparoscopic electrodes. As used herein, the term “electrosurgery instrument” is meant to refer to any surgical instrument, or portion of surgical instrument, that includes an electrode adapted to deliver RF current to tissue for the purpose of cutting, coagulating, and/or desiccation, such as electrosurgery blades, electrosurgery forceps, and laparoscopic electrodes, with or without the pencil body, cabling, RF generator, and other components.

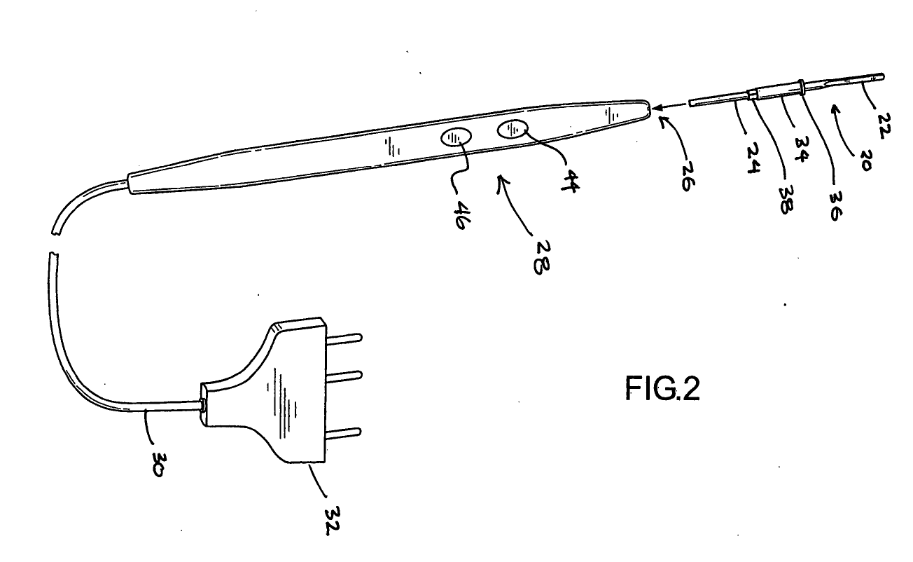

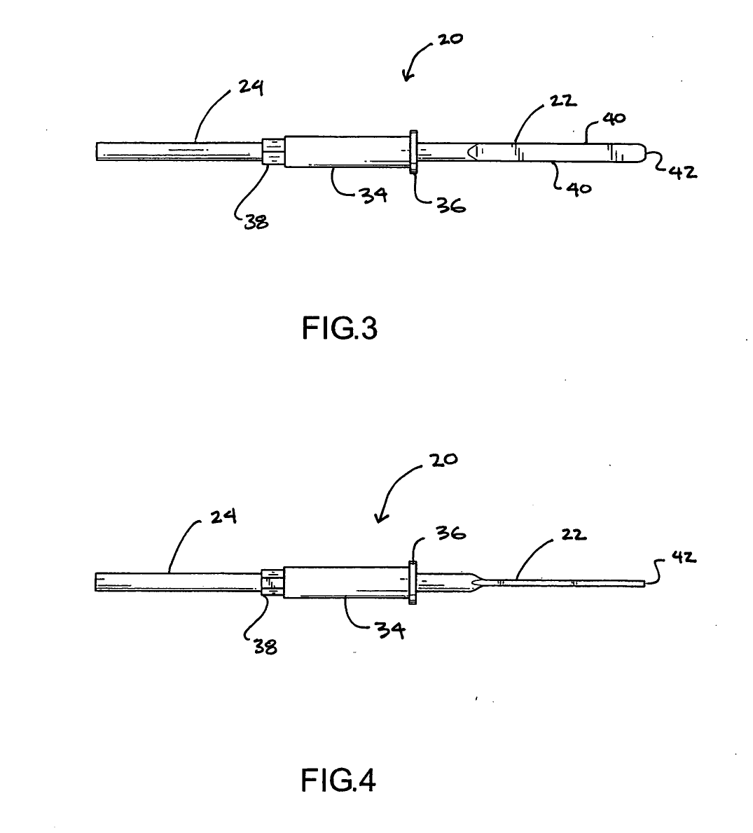

FIG. 2 is an isometric view of an electrosurgery blade 20 (also referred to as an “electrode knife”) conforming to aspects of the present invention. FIG. 3 is a side view of the blade of FIG. 2, and FIG. 4 is a top view of the blade of FIG. 4. Referring to FIGS. 2-4, the electrosurgery blade includes a NiTi-SMA cutting region 22 formed at a distal end of the blade. The cutting region may also be referred to as an electrode. The cutting region or electrode is the portion of the blade that delivers RF energy to the target tissue region during a surgical procedure. The electrode may define any number of shapes, some of which are shown in various embodiments discussed herein. In one sense, aspects of the invention include any electrosurgery blade including a NiTi-SMA electrode or cutting region, regardless of the shape and size of the cutting region, and regardless of the shape and configuration of other portions of the blade.

The blade further includes a shaft 24 extending from the cutting region to a proximal end region of the blade. The shaft is electrically conductive. The proximal end region of the shaft is adapted to be inserted into a socket 26 formed in the distal end of an electrosurgery pencil 28. The pencil has a cable 30 with a plug 32 adapted to plug into an RF generator (not shown). Inserted in the pencil, electrosurgical current may be applied to the shaft 24 and conveyed to the electrode 22. An insulating sleeve 34 is located along a portion of the blade 20 between its ends. The distal end region of the sleeve includes a circumferential longitudinally extending flange 36. Generally, the sleeve is fabricated from an electrically insulating material, and may be a spray-on coating, shrink tubing, in-molded or molded on, press fit, or otherwise secured on a portion of the electrode and/or shaft. The insulating material may extend along any length of the electrode. The sleeve 34 and flange 36 help prevent inadvertent tissue contact by an energized electrode. The proximal end region of the sleeve defines a reduced diameter multifaceted region 38 adapted to fit into a corresponding aperture of some model pencils 28. The facets (or flat sides) help prevent the blade from rotating in the pencil during use.

Referring to the side view of FIG. 3, the electrode 22 defines a generally rectangular area. The electrode also defines a generally rectangular cross section, with opposing sides and opposing cutting edges 40. The cutting region defines a blunt tip 42 at its distal end. The cutting edges are substantially narrower than the sides. During an electrosurgery procedure, either the top or bottom cutting edge is placed in contact with or in close proximity to the target tissue in order to cut the tissue or coagulate. Cutting occurs when the cutting edge is placed adjacent the target tissue, and the blade is energized with the appropriate current. Typically, current is applied continuously in order to cut tissue. The tissue adjacent the blade separates leaving a well-defined incision. The tissue does not separate from contact with the cutting edge, as is the case with a traditional scalpel. Typically, the cutting edge of an electrosurgical blade is fairly dull and cannot conventionally cut tissue in a satisfactory manner. The separated tissue passes by the sides of the electrode as the surgeon guides the blade, while the electrical energy creates the incision. Coagulating occurs similarly to cutting, but with a different current than cutting. The blade is brought into close proximity with the bleeding tissue and energized appropriately. Typically, coagulation occurs by cycling the current on and off at the appropriate frequency. Simultaneously cutting and coagulating tissue with an electrosurgery blade occurs by blending the coagulation current with a continuous cutting current. A typical electrosurgery pencil includes a “cut” button 44 and a “coag” button 46 adapted to deliver cutting energy and coagulating energy, respectively.

Advantageously, the electrode 22 is formed of NiTi-SMA (e.g., Nitinol®). Testing performed by the applicant indicates that a NiTi-SMA cutting region is highly resistant to eschar buildup during surgical procedures. Additionally, in most forms NiTi-SMA is biocompatible. As such, a blade 20 including a NiTi-SMA electrode 22, conforming to aspects of the present invention, is useful in performing electrosurgery procedures and at the same time avoiding the problematic buildup of eschar on the electrode. As mentioned above, the shaft 24 and electrode region 22 may be formed of NiTi-SMA, or the electrode region may be formed of NiTi-SMA and the shaft region formed of some other material. In such a case, the electrode is electrically coupled to the shaft in a manner so that current may be delivered to the electrode from the RF generator.

In one particular embodiment where the cutting region and shaft are each NiTi-SMA, the cutting region 22 is formed by grinding an NiTi-SMA wire. The wire defines the diameter of the shaft 24 and is originally fabricated of the appropriate diameter to engage a particular pencil. One particular embodiment is fabricated with a 0.094 inch Nitinol wire with 54.5% to 56.1% Nickel by weight. The sides of the cutting region are each ground on an appropriate tool, such as a mechanical grinder or wire EDM (“Electrical Discharge Machine”). When the sides are ground, the cutting edges 40 are defined by the intersection of material between the tops and bottoms of each side. After grinding, the sides and/or cutting edges may be polished in a mechanical polishing procedure. Polishing is not required, although it may further help in avoiding eschar buildup on the NiTi-SMA electrode.

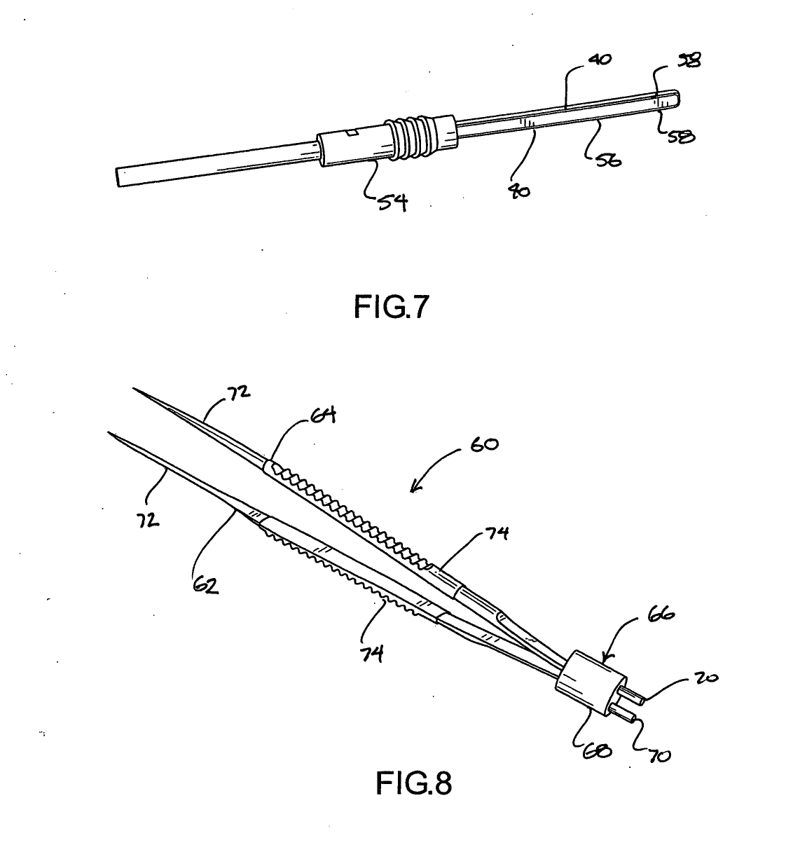

FIGS. 5-7 illustrate alternative electrosurgery blades, conforming to aspects of the present invention. The embodiments of FIG. 5 and FIG. 7 are similar to that shown in FIGS. 2-4, with some differences. The embodiment of FIG. 5 includes a differently shaped sleeve 48 than that of FIGS. 2-4. The sleeve of the FIG. 5 blade does not include the circumferential flange or the reduced diameter sleeve region 34 shown in the sleeve of FIGS. 2-4. Further, the distal end of the sleeve is closer to the cutting region, defining a shorter cutting region 50. The sleeve of FIG. 5 may be formed of electrically insulating heat shrink tubing. The width of the cutting region, between cutting edges 40, is slightly wider than the shaft because the cutting region is stamped NiTi-SMA wire rather than a ground NiTi-SMA wire. Stamping may not be suitable for all types of NiTi-SMAs.

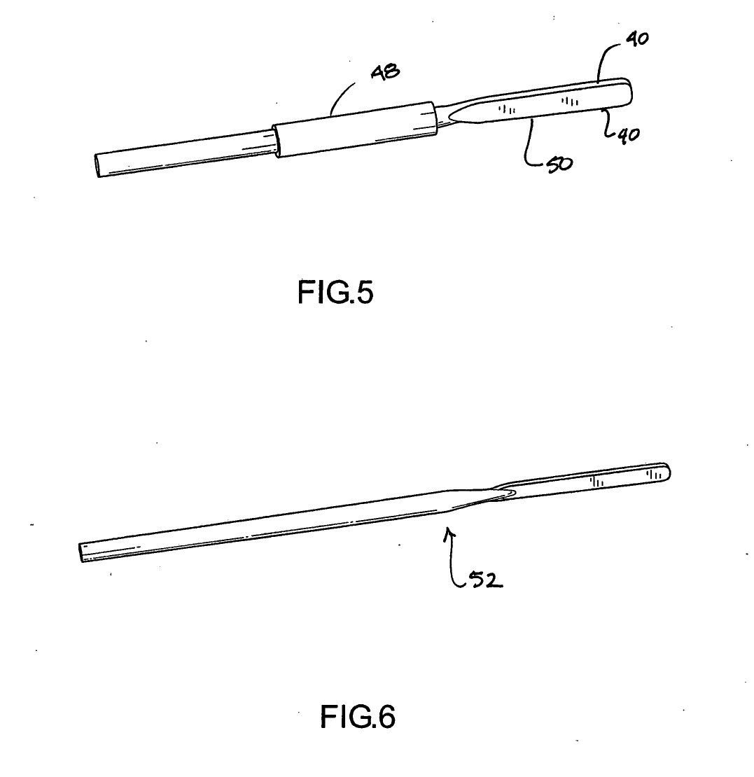

The embodiment of FIG. 6 shows a blade 52 without a sleeve. A sleeve is nearly always included with a blade during a procedure; however, it is possible to provide blades without a sleeve, or with various sleeves that a surgeon may add to the blade before a procedure. The embodiment of FIG. 7 includes a differently shaped sleeve 54 and a differently shaped NiTi-SMA cutting region 56, than other embodiments. The cutting region defines beveled edges 58 between the sides and cutting edges 40. The beveling can help increase current density at the cutting edge, while maintaining a suitably thick cutting region that resists substantial flexing and heating during use. Increased current density at the cutting edge can increase cutting efficiency in some instances.

FIG. 8 illustrates an alternative electrosurgery instrument conforming to aspects of the present invention. The electrosurgery instrument of FIG. 8 is a forceps 60 having a first pincer 62 and a second pincer 64. A plug 66 is situated at the proximal end region of the forceps. The plug includes an insulated plug body 68. Plug pins 70 electrically coupled to each pincer extend from the body. The forceps is adapted to be plugged into an appropriate RF generator in order to apply current to each pincer. More particularly, each pincer includes a NiTi-SMA tip region 72 at the distal end of each pincer. The NiTi-SMA tip regions are electrically connected with the corresponding plug pins. Between the plug and the tip region of each pincer is an insulated gripping region 74. During a medical procedure, a surgeon grasps the opposing gripping regions of the pincers and manipulates the forceps appropriately. When necessary, the surgeon applies RF energy to the opposing tip regions of the pincers in order to cut tissue or coagulate a target tissue.

In one particular implementation, each pincer comprises a NiTi-SMA section extending between the plug and the tip region, rather than discrete NiTi tip regions, although discrete NiTi tip regions are possible with the remaining pincer length being a different material. Moreover, NiTi tip attachments may be fit over conventional stainless steel forceps pincer tips. The insulating gripping region comprises a sleeve placed over the NiTi-SMA pincer core. The sleeve may include ridges or other raised surfaces adapted to enhance the grip of the pincers. Further, the sleeve may be shaped so it does not rotate on the pincer during use.

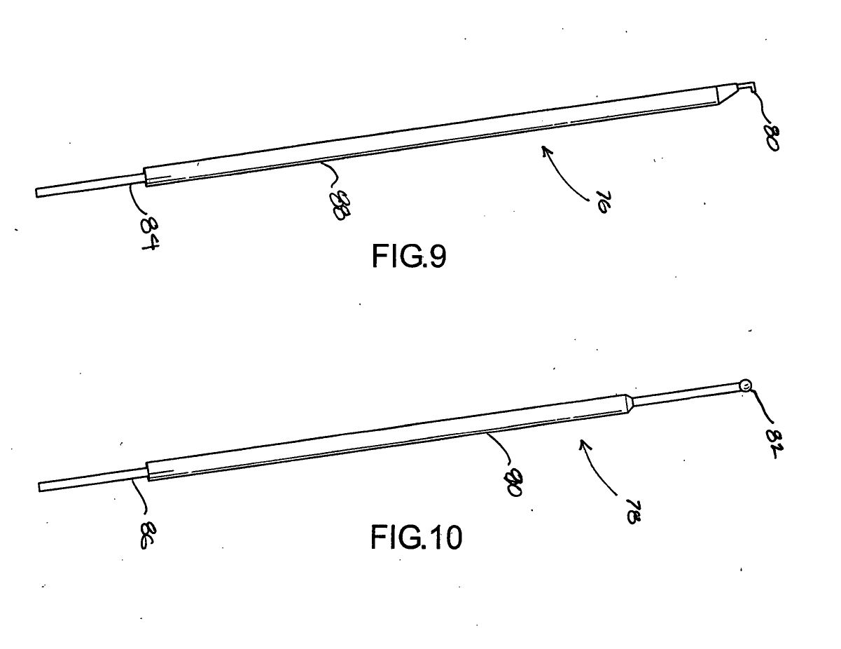

FIGS. 9 and 10 illustrate isometric views of two laparoscopic electrode type electrosurgery instruments (76, 78), having differently shaped NiTi electrodes (80, 82). Both laparoscopic instruments include elongate shafts (84, 86) shrouded in an electrically insulated sleeve (88, 90). The proximal end of the shaft extends from the sleeve and is adapted to be plugged into a pencil in a similar manner as an electrosurgery blade. An electrode region of the shaft extends from the distal end of the sleeve. The electrode (80, 82) is in electrical communication with the proximal end of the shaft (84, 86), and may be a contiguous extension of the shaft or electrically connected with the shaft in some manner. In the FIG. 9 embodiment, the electrode region 80 is fabricated with a NiTi alloy shaped into a hook. In the FIG. 10 embodiment, the electrode region 82 is fabricated with a NiTi alloy shaped into a ball. Other shapes are possible, such as needle shapes, curved elongate rectangle shapes, spatula shapes, and various length and shaped hooks.

Besides NiTi-SMA wire, it is also possible to fabricate relevant electrode portions of an electrosurgical instrument conforming to aspects of the present invention (e.g., electrode, forceps, forceps pincers, tip region of pincers, electrode balls, hooks, needles, spatulas, etc.) with NiTi-SMA ribbon, microtubing, sheets, rods, bars, and other base forms. Some base NiTi-SMA forms might facilitate fabrication of a desired shape more readily than other base forms. Additonally, it is possible to wrap or adhere NiTi-SMA to an underlying base material. For example, a cutting region of a blade can include NiTi sheet or ribbon material on a stainless steel base or core, properly dimensioned. Moreover, it may be possible to directly mold NiTi-SMA in the desired form.

Although various representative embodiments of this invention have been described above with a certain degree of particularity, those skilled in the art could make numerous alterations to the disclosed embodiments without departing from the spirit or scope of the inventive subject matter set forth in the specification and claims. All directional references (e.g., upper, lower, upward, downward, left, right, leftward, rightward, top, bottom, above, below, vertical, horizontal, clockwise, and counterclockwise) are only used for identification purposes to aid the reader's understanding of the embodiments of the present invention, and do not create limitations, particularly as to the position, orientation, or use of the invention unless specifically set forth in the claims. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

In some instances, components are described with reference to “ends” having a particular characteristic and/or being connected to another part. However, those skilled in the art will recognize that the present invention is not limited to components which terminate immediately beyond their points of connection with other parts. Thus, the term “end” should be interpreted broadly, in a manner that includes areas adjacent, rearward, forward of, or otherwise near the terminus of a particular element, link, component, member or the like. In methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

We claim:

1. An electrosurgery instrument for conducting electrical energy to tissue during an electrosurgery procedure, the electrosurgery instrument comprising:
a conductive shaft region arranged to receive electrical energy;
an electrode in electrical communication with the conductive shaft; and
wherein the electrode is fabricated with a Nickel Titanium alloy.

2. The electrosurgery electrode of claim 1 wherein the electrode defines opposing sides and opposing cutting edges.

3. The electrosurgery electrode of claim 1 wherein the electrode defines a ball.

4. The electrosurgery electrode of claim 1 wherein the electrode defines a hook.

5. The electrosurgery electrode of claim 1 wherein the electrode defines a spatula.

6. The electrosurgery electrode of claim 1 wherein the electrode defines a needle.

7. The electrosurgery instrument of claim 1 further comprising a pencil having a socket for receiving the conductive shaft.

8. The electrosurgery instrument of claim 1 further comprising an electrically insulating sleeve arranged over a portion of the electrode.

9. The electrosurgery instrument of claim 1 wherein the electrosurgery instrument is an electrosurgery blade.

10. The electrosurgery instrument of claim 1 wherein the electrosurgery instrument is a laparoscopic instrument.

11. The electrosurgery instrument of claim 1 wherien the electrode is fabricated with a Nickel Titanium shape memory alloy.

12. The electrosurgery instrument of claim 1 wherein the electrode is an extension of the conductive shaft.

13. An electrosurgery forceps comprising:
a first pincer including a first Nickel Titanium alloy tip region; and
a second pincer including a second Nickel Titanium alloy tip region, the second pincer operably coupled with the first pincer.

14. The electrosurgery forceps of claim 13 wherein:
the first pincer includes an insulating sleeve; and
the second pincer includes an insulating sleeve.

15. The electrosurgery forceps of claim 13 wherein:
the first pincer is fabricated with Nickel Titanium alloy; and
the second pincer is fabricated with Nickel Titanium alloy.

16. The electrosurgery forceps of claim 15 wherein:
the first tip region is a contiguous portion of the first pincer; and
the second tip region is a contiguous portion of the second pincer.

17. The electrosurgery forceps of claim 13 further comprising a plug including a first prong electrically coupled with the first tip region and a second prong electrically coupled with the second tip region.

18. The electrosurgery forceps of claim 13 wherein the first pincer includes a first Nickel Titanium shape memory alloy tip region; and
the second pincer includes a second Nickel Titanium shape memory alloy tip region.

19. An electrosurgery instrument comprising:
means for electrically coupling the instrument with a pencil;
means for delivering radio frequency energy to a target tissue region; and
the means for delivery being at least partially fabricated with a Nickel Titanium alloy.
